(19) United States
(12) Patent Application Publication
(10) Pub. No.: US 2026/0053989 A1
(43) Pub. Date: Feb. 26, 2026

(54) GEL COMPOSITION FOR XENOTRANSPLANTATION OR ALLOTRANSPLANTATION AND MANUFACTURING METHOD OF THE SAME

(71) Applicant: TZU CHI UNIVERSITY, HUALIEN CITY (TW)

(72) Inventor: Chin-Yu LIN, HUALIEN CITY (TW)

(21) Appl. No.: 19/009,623

(22) Filed: Jan. 3, 2025

(30) Foreign Application Priority Data
Aug. 20, 2024 (TW) 113131262

(51) Int. Cl. A61L 27/56; A61L 27/26; A61L 27/36; A61L 27/38; A61L 27/52; C08L 5/04; C08L 89/06; C12N 9/64

(52) U.S. Cl. CPC A61L 27/56; A61L 27/26; A61L 27/3687; A61L 27/3804; A61L 27/52; C08L 5/04; C08L 89/06; C12N 9/6416; A61L 2300/64; A61L 2430/40; C12Y 304/24003

(57) ABSTRACT

The present disclosure provides a gel composition comprising: a porous scaffold and a gel coating. The porous scaffold is filled with a biological tissue or a biological cell, and the gel coating covers the porous scaffold. The present disclosure further provides the manufacturing method and the use of the foregoing gel composition, especially the use as an implant for xenotransplantation or allotransplantation.

### FIELD OF THE INVENTION

The disclosure is involved in the field of regenerative medicine, and particularly directed to a gel composition for xenotransplantation or allotransplantation and manufacturing method of the same.

### BACKGROUND OF THE INVENTION

The parathyroid gland is an endocrine system located posteriorly to the thyroid gland, responsible for secreting parathyroid hormone to modulate the calcium and phosphorous homeostasis in the blood and skeletal bones. Calcium is a vital physiological element modulated by parathyroid hormone, vitamin D, and calcitonin, influencing neural stimulation, cell movement, signal transduction, and enzyme activity.

Furthermore, the blood calcium concentration possesses a feedback effect, controlling the parathyroid hormone released from the parathyroid gland. In blood calcium in a low-level circumstance, the parathyroid gland releases the parathyroid hormone to stimulate the bone, kidney, and intestine to release calcium into the blood and increase the phosphorous excretion from urine, and eventually maintain the balance of calcium and phosphorous concentration in blood. Therefore, low-level blood calcium is often accompanied by high-level blood phosphorous.

Principally, the parathyroid hormone activates the osteoclast to digest bone, release calcium into the blood, and promote the kidney and intestine to resorb the calcium while diminishing the calcium catabolism. Patients with hypoparathyroidism usually suffer low-level blood calcium and elevated phosphorous levels in serum accompanied by insufficient parathyroid hormone secretion. Although hypoparathyroidism is a rare disease in the human population, there is a lack of formal counting for the rate in most countries. Generally, 23 to 37 cases in every 100 thousand people, most cases happening in older women and patients with thyroid gland resection. Accordingly, hypoparathyroidism resulting from a congenital abnormality, autoimmune disease, or neck and thyroid resection elicits chronic and acute parathyroid gland malfunction, which accounts for approximately 75% of hypoparathyroidism's rate. Clinically, acute hypoparathyroidism elicits neural, muscular, and heart failure, which may lead to death, while chronic hypoparathyroidism elicits low-level blood calcium and may also lead to vision impairment. Therefore, medication, organ transplantation, and cell therapy addressed to compensate for insufficient parathyroid hormone secretion have emerged.

Acute therapy uses calcium gluconate IV injection for emergency treatment, followed by oral calcium supplements or high-dose vitamin D for long-term medication. However, the long-term medication for the treatment of hypoparathyroidism is unsatisfactory due to the drugging dose for individual patients is uncontrollable. Meanwhile, patients are usually accompanied by other diseases, leading to ineffective treatments and subsequent symptoms of hyperphosphatemia and hypercalciuria, which may lead to kidney impairment eventually. Meanwhile, current medication also cannot precisely and immediately control the calcium level in the blood.

These urgent unmet medical needs promote the development of new therapies for hypoparathyroidism, such as parathyroid gland allotransplantation, which still has immune rejection problem and leads to transplantation failure. A developing engineering methodology utilizing the concept of cell encapsulation for allogenic or xenogenic transplantation maintains the cell viability physiological functions, exchanges the cell proteins, and remarkably prevents the immune cell recognition and attack.

### SUMMARY OF THE INVENTION

The first embodiment of the disclosure provides a gel composition comprising: a porous scaffold filled with a biological tissue or a biological cell; and a gel coating covering the porous scaffold.

In the above-mentioned gel composition, the gel coating may comprise one or more of agar, agarose, alginic acid, alguronic acid, α-glucan, amylopectin, amylose, arabinoxylan, β-glucan, callose, carrageenan polysaccharides, cellodextrin, cellulin, cellulose, chitin, chitosan, chrysolaminarin, curdlan, cyclodextrin, α-cyclodextrin, dextrin, glucan, ficoll, fructan, fucoidan, galactoglucomannan, galactomannan, galactosaminoogalactan, gellan gum, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, schizophyllan, sinistrin, sizofiran, welan gum, xanthan gum, xylan, xyloglucan, and zymosan; preferably, the gel coating comprises alginic acid.

In the above-mentioned gel composition, the porous scaffold may comprise one or more of a porous polymeric particle or matrix, a porous inorganic particle or matrix, and an etched metal particle or matrix; preferably, the porous polymeric particle or matrix comprises one or more of chitosan, hyaluronic acid, gelatin, collagen, chitosan, poly(lactic-co-glycolic acid) (PLGA), polycyclohexylenedimethylene terephthalate (PCT), the porous inorganic particle or matrix comprises one or more of silicon dioxide and tricalcium phosphate, and the etched metal particle or matrix comprises titanium; more preferably, the porous polymeric particle or matrix comprises gelatin.

In the above-mentioned gel composition, the biological tissue may comprise one or more of a pancreatic islet tissue, a pancreatic duct tissue, a liver tissue, a nervous tissue, a thyroid gland tissue, a parathyroid gland tissue, a kidney tissue, an adrenal gland tissue, a pituitary gland tissue, a spleen tissue, an adipose tissue, and a myeloid tissue; preferably, the biological tissue comprises a parathyroid gland tissue.

In the above-mentioned gel composition, the biological cell may comprise one or more of a pancreatic islet cell, a pancreatic duct cell, a liver cell, a nervous cell, a thyroid gland cell, a parathyroid gland cell, a kidney cell, an adrenal gland cell, a pituitary gland cell, a spleen cell, an adipose cell, and a myeloid cell; preferably, the biological cell comprises a parathyroid gland cell.

In the above-mentioned gel composition, the gel coating may comprise alginic acid, the porous scaffold may comprise gelatin, the biological tissue may comprise a parathyroid gland tissue, and the biological cell may comprise a parathyroid gland cell.

The second embodiment of the disclosure provides a method for producing the above-mentioned gel composition, comprising: filling the porous scaffold with the biological tissue or the biological cell; immersing the porous scaffold in a gel precursor solution; and adding a crosslinker solution into the gel precursor solution immersed with the porous scaffold to perform cross-linking reaction so as to form the gel coating, the gel coating covering the porous scaffold.

In the above-mentioned producing method, the crosslinker solution may comprise one or more of a calcium ion, a barium ion, and a zinc ion; preferably, the crosslinker solution comprises a barium ion.

In the above-mentioned producing method, before filling the porous scaffold with the biological tissue or the biological cell, the biological tissue or the biological cell is digested or dissociated with an enzyme; preferably, the enzyme is a type II collagenase.

The third embodiment of the disclosure provides a method for xenotransplantation or allotransplantation, comprising: implanting the above-mentioned gel composition into a subject in need of xenotransplantation or allotransplantation.

In accordance with the disclosure, the gel composition can be implanted into a recipient for xenotransplantation or allotransplantation so that the biological tissue or the biological cell contained in the gel composition not only can exhibit its original biofunction, but also be prevent from the happening of foreign body reaction and inflammatory cell attack.

### BRIEF DESCRIPTION OF THE DRAWINGS

FIG. 1A is a fluorescence microscopy image showing the parathyroid tissue stained with FDA and PI;

FIG. 1B is a bar chart illustrating the signal intensity of the parathyroid tissue stained with FDA and PI;

FIG. 1C is a bar chart illustrating the viability of the parathyroid tissue at specific day after in vitro culture compared with cells at day 0;

FIG. 1D is a fluorescence microscopy image showing the parathyroid tissue stained with FDA and PI after incubation with calcium ion;

FIG. 2A is a flow chart illustrating the in vitro preparation of the parathyroid tissue and the parathyroid cells;

FIG. 2B is a flow chart illustrating the preparation of the PTH gland tissue loaded AM and the PTH cells loaded AM;

FIG. 2C is a flow chart illustrating the preparation of the PTH cells loaded gel-spon-AM;

FIG. 3 is an image showing the appearance of the PTH gland tissue loaded AM, the PTH cells loaded AM, and the PTH cells loaded gel-spon-AM;

FIG. 4A is an image and a scanning electron microscopic image showing the appearance and the interior of the PTH cells loaded AM and the PTH cells loaded gel-spon-AM;

FIG. 4B is a curve chart illustrating the biomechanical properties of the PTH cells loaded AM and the PTH cells loaded gel-spon-AM;

FIG. 5A is a bar chart illustrating the PTH released from the parathyroid cells loaded AM followed with the calcium ion concentration adjusted;

FIG. 5B is a bar chart illustrating the PTH released from the parathyroid cells loaded gel-spon-AM followed with the calcium ion concentration adjusted;

FIG. 6A is a fluorescence microscopy image showing the parathyroid cells loaded AM and the parathyroid cells loaded gel-spon-AM stained with FDA and PI;

FIG. 6B is a bar chart illustrating the signal intensity of the parathyroid cells loaded AM and the parathyroid cells loaded gel-spon-AM stained with FDA and PI and the total protein amount thereof;

FIG. 7A is a bar chart illustrating the CaSR protein expression level of the parathyroid cells encapsulated in different forms;

FIG. 7B is a bar chart illustrating the GCM2 transcription factor expression level of the parathyroid cells encapsulated in different forms;

FIG. 8A is an image showing the appearance of the parathyroid cells encapsulated in different forms in subcutaneous implantation;

FIG. 8B is a tissue staining image showing the parathyroid cells encapsulated in different forms stained with HE after retrieval from subcutaneous implantation;

FIG. 9A is an image showing the appearance of the parathyroid cells encapsulated in different forms after retrieval from intraperitoneal implantation;

FIG. 9B is a tissue staining image showing the parathyroid cells encapsulated in different forms stained with HE after retrieval from intraperitoneal implantation;

FIG. 10A is an immunohistochemistry image showing the parathyroid cells encapsulated in different forms immunostained with CD4 antibody after retrieval from subcutaneous implantation;

FIG. 10B is an immunohistochemistry image showing the parathyroid cells encapsulated in different forms immunostained with CD68 antibody after retrieval from subcutaneous implantation; and

FIG. 10C is an immunohistochemistry image showing the parathyroid cells encapsulated in different forms immunostained with α-SMA after retrieval from subcutaneous implantation.

### DETAILED DESCRIPTION OF THE INVENTION

The detailed description and preferred embodiments of the invention will be set forth in the following content, and provided for people skilled in the art to understand the characteristics of the invention.

Alginic acid is a natural, biocompatible, and biodegradable polysaccharide crosslinked by metal bivalent cation to form an alginate hydrogel with adjustable porosity. Cells can be trapped in the alginate network covalently linked by the contiguous β-D-mannuronate (M) and α-L-guluronate (G) residues. M/G ratio, copolymer length, molecular weight, and viscosity dominate the physicochemical performance, such as alginate with higher G content possesses superior biocompatibility, meet the requirement for cell encapsulation. Presently, many studies utilize alginate in cell protection and delivery for disease treatment, such as acute liver failure therapy using alginate-encapsulated human hepatocytes and insulin insufficiency using islet cells loaded alginate beads.

Optimal scaffold plays a vital role in a successful tissue engineering approach, serving as the carrier for drug and cell delivery and providing the matrix for cell adhesion, proliferation, and differentiation. The previous publication uses a hydrogel for an organoid study, but the nutrition and waste exchange are inefficient due to the scanty 3D space for cell growth and weak biomechanical property. Furthermore, the smooth surface of microbeads and limited space for transplanted cell adhesion and proliferation eventually lead to inferior cell viability and therapeutic efficiency. Therefore, creating a circumstance that provides cell survival, metabolism, and proliferation is essential to a successful cell transplantation.

Gelatin is a non-toxic, broadly studied biopolymer applied in regenerative medicine with a chemical property similar to the extracellular matrix (ECM) in natural tissues, supporting cell proliferation and differentiation and high biocompatibility, eliciting deficient immune responses after grafting in vivo. A previous study using the gelatin and alginate composite scaffold efficiently promotes the chondrogenesis of human adipose-derived stem cells and maintains cell viability and extracellular matrix expression.

Therefore, in this disclosure, it is attempted to develop a secretory cell transplantation methodology that protects the transplanted cells from immune attack while maintaining the inherent endocrine function of the transplanted cells, simultaneously releasing the hormone. The disclosure provides an alginate microbead manufacture methodology for the delivery of parathyroid cells, compared the alginate microbeads crosslinked by calcium or barium ions, and also pre-loaded parathyroid cells in a gelatin sponge, followed with alginate encapsulation for the manufacture of gelatin sponge-alginate composite microbeads. Furthermore, microbeads' biomechanical properties, endocrine release, and cell proliferation are examined in vitro. Subsequently, the biocompatibility and protection capability of the microbead after implantation in the immunocompetent mice in vivo are also evaluated.

The following examples are offered to further illustrate the invention.

### Example 1: Dissociation of the Parathyroid Gland and Preparation of Conventional AM and Gel-Spon-AM

Parathyroid glands were collected from patients accepting the parathyroidectomy in China Medical University Hospital under the approval of IRB and complete consensus from patients. The fresh parathyroid glands removed from patients were immediately transferred to the P2 lab, washed, and dissected into approximately 2 mm×2 mm×2 mm cubes through aseptic manipulation. To collect the parathyroid cells, the dissected parathyroid gland cubes were incubated with an equal volume of 0.1% type II collagenase dissolved in serum-free DMEM/Ham's F-12 medium for 60 minutes in 37° C. The type II collagenase digestion was neutralized by equal volume of complete RPMI 1640 medium containing 10% FBS, filtrated by 70-mesh, centrifuged at 346 g, 5 minutes, and washed three times.

To prepare the parathyroid tissue loaded alginate microbeads (AM), an 8-mesh filtered parathyroid tissue cube was immersed in 50 μL 1.5% sodium alginate solution, dropped into 10 mM CaCl₂) or BaCl₂ solution, inverted and stood 2 minutes, washed with PBS thoroughly, and transferred to RPMI 1640 complete medium for 37° C., 5% CO₂ culture. Similarly, to prepare the parathyroid cells loaded microbeads, the collagenase-digested cells were manipulated in sodium alginate solution, CaCl₂), or BaCl₂ solution, with the procedures identical to preparing the tissue loaded microbeads.

To prepare the parathyroid cells loaded gelatin sponge alginate microbeads (gel-spon-AM), a 7 μL RPMI 1640 medium containing 1×10⁴ cells were dropped into sterilized gelatin sponge, stood 5 minutes, and transferred to 50 μL 1.5% sodium alginate solution, subsequently dropped into 10 mM CaCl₂) or BaCl₂ solution in 50 mL tube, inverted and stood 2 minutes, and cultured in 37° C., 5% CO₂ for subsequent use.

### Example 2: SEM and Biomechanical Testing

The inner structure of microbeads was examined by scanning electron microscopy. Briefly, microbeads were fixed by glutaraldehyde freeze-dried by vacuum lyophilizer for 48 hours. Before electron microscopic observation, lyophilized AM or gel-spon-AM samples were attached on double-sided conductive tape, which was adhered to a specimen stub and coated with gold for 40 sec using a sputter coater to induce electrical conductivity to the samples. To examine the compressing biomechanical property of microbeads after culturing in a complete medium for 3, 7, and 14 days, the microbeads were collected from the medium, immediately dried with tissue paper, and transferred onto a new 3M filter paper, mounted on the testing stage of a compressing machine. The loading speed is 1 mm per minute, and the maximum distance and axial force are 5 mm and 8N, respectively. Data was immediately recorded until the microbead broke, and axial force sharply returned to the ground level.

### Example 3: Live-and-Dead Test of Parathyroid Gland Tissues and Cells

The dissected parathyroid gland cubes and parathyroid cells loaded AM and gel-spon-AM at different culture periods were removed, washed with PBS, and immediately incubated with FDA (8 μg/mL) and PI (20 μg/mL) dissolved in PBS for 5 minutes at dark space. Subsequently, the staining solution was removed, washed and supplemented with PBS, and observed through confocal microscopy. The contour of parathyroid tissue or cells loaded microbead was circled as the region of interest (ROI) for fluorescent intensity quantification by ImageJ. The following equation evaluated the tissue and cell viability:

$$viability ( \% ) = \frac{mean fluorescent intensity of FDA}{mean fluorescent intensity of FDA + PI} \times 100 \%$$

### Example 4: Calcium Stimulation and Parathyroid Hormone Release Measurement

To examine the healthy parathyroid tissues or cells loaded microbeads respond to the stimulation from surrounding calcium ions to secrete the parathyroid hormone, a cyclic high and low calcium level of the immersing experiment was processed. The parathyroid tissues or cells-loaded microbeads were cultured in complete RPMI 1640 medium overnight and changed to new RPMI 1640 medium containing 1.3 mM CaCl₂) for 16 hours at 37° C., 5% CO₂ for subsequent high- and low-level calcium stimulation. Firstly, the microbeads were washed with HBSS and immediately transferred to the following 2 mL RPMI 1640 medium containing 1.3 mM, 0.42 mM, 2.6 mM, 0.42 mM, 2.6 mM and 0.42 mM CaCl₂), respectively, in a 6-well plate. Each step was incubated for 30 minutes and washed with HBSS at every transfer. The medium was collected for PTH ELISA measurement.

### Example 5: Proliferation Test of Parathyroid Cells Loaded Microbead

The proliferation capability of parathyroid cells loaded in conventional alginate microbead (AM) or gelatin sponge-alginate composite microbead (gel-spon-AM) was examined by total protein content in microbead. Briefly, the cells-loaded microbead was dissociated by 0.5 M EDTA, centrifuged at 4000 rpm for 5 minutes, and washed with PBS twice. Cells were digested by 30 mL RIPA, vortex at 4° C. for 30 minutes, and centrifuged at 12000 rpm. The supernatant was collected for total protein measurement using the BCA Protein Assay Kit and measured the absorbance at 562 nm by a microplate reader.

### Example 6: Western Blot

To analyze the calcium-sensing receptor (CaSR) and Glial cells missing-2 (GCM2) protein expression in parathyroid cells encapsulated in the microbeads, the collected cells were subjected to protein extraction and western blot by standard protocol. Briefly, cell proteins were extracted with RIPA lysis buffer containing 0.2% phosphatase inhibitor cocktail (Cat. P2850, Sigma-Aldrich, USA), phenylmethylsulfonyl fluoride at 4° C. for one hour, 15,000 rpm centrifugation 15 minutes for protein quantification using Pierce™ BCA Protein Assay Kit. Proteins (10 μg) were electrophoresed by SDS-polyacrylamide gel and transferred to a PVDF (poly(vinylidene fluoride)) membrane. The membrane was blocked in 5% fat-free milk in PBST (PBS with 0.05% Tween-20), followed by incubation overnight with the following primary antibodies diluted in PBST: CaSR (1:1000), GCM2 (1:1000) and GAPDH (1:1000). The primary antibodies were removed, and the membrane was washed extensively in PBST. Subsequent incubation with horseradish peroxidase-conjugated goat anti-mouse antibodies (1:10,000) was performed at room temperature for two hours. The membrane was washed extensively in PBST to remove excess secondary antibodies, and the blot was visualized with enhanced chemiluminescence reagent.

### Example 7: Animal Implantation

The animal study was conducted with the approval of the animal study committee board. The 8- to 10-week-old ICR mice were anesthetized by inhalation of 2.5% isoflurane. For subcutaneous implantation of microbeads, the mouse was placed in a prone position, a 1.0 to 1.5 cm sagittal incision was made on the lateral back skin, adjacent to the hind limb, and exposed the subcutaneous pocket between the skin and lower muscle facia layer. A microbead was placed in the pocket, and the skin lesion was closed by a 4-0 nylon suture, a few gentamycin-containing ointments, and medicated with 10 mg/kg acetaminophen in drink water for three days. A similar operation was proceeded for the intraperitoneal implantation of microbeads in mice. Briefly, the mouse was placed supine, and a 1.0 to 1.5 cm sagittal incision was made on the skin of the parietal peritoneum behind the vertebrochondral ribs, approximately 1 cm. A microbead was placed in the peritoneal cavity, and the peritoneum and skin lesion were closed by 6-0 Ethicon VICRYL absorbable and 4-0 nylon non-absorbable suture, respectively. The lesion was spread with a few gentamycin-containing ointments and medicated with 10 mg/kg acetaminophen in drink water for three days.

### Example 8: Histological Examination

Mice were sacrificed at the indicated date as depicted in the Results; the microbeads retrieved from the subcutaneous and intraperitoneal implantations were collected, fixed with 4% paraformaldehyde in PBS for three days, embedded in OCT, and serially frozen-sectioned in 5 μm thickness for H&E and immunohistochemical staining by standard protocol. Briefly, slides were washed with PBS, blocked with 1% BSA, and immunostained with primary antibody including rabbit anti-mouse CD4 (1:100), rabbit anti-mouse CD68 (1:100) and rabbit anti-mouse α-SMA (1:100) diluted in 1% BSA in PBS containing 100 mM Triton-X 100 at 4° C. overnight. Subsequently, the slides were washed three times with PBS containing 100 mM Triton-X 100, then stained with Alexa 488-conjugated goat anti-rabbit secondary antibody at room temperature for one hour, and counterstained with DAPI. The fluorescent IHC staining was observed using a fluorescence microscope. The histomorphometric analysis of IHC staining was quantified by three independent pathologists and circled the region of interest (ROI) by ImageJ Fiji.

### Example 9: Statistical Analysis

Data are presented as means±SDs, statistical comparisons were performed by Student's t-test or one-way analysis of variance (ANOVA), and p values <0.05 were considered significant. All calculations were performed using a Statistics Analysis System licensed to China Medical University. All in vivo data represent at least three independent experiments, as indicated.

Result 1: Clinical Parathyroid Cells Show High Viability with a Superior PTH Secretory Ratio than Parathyroid Tissue Pieces

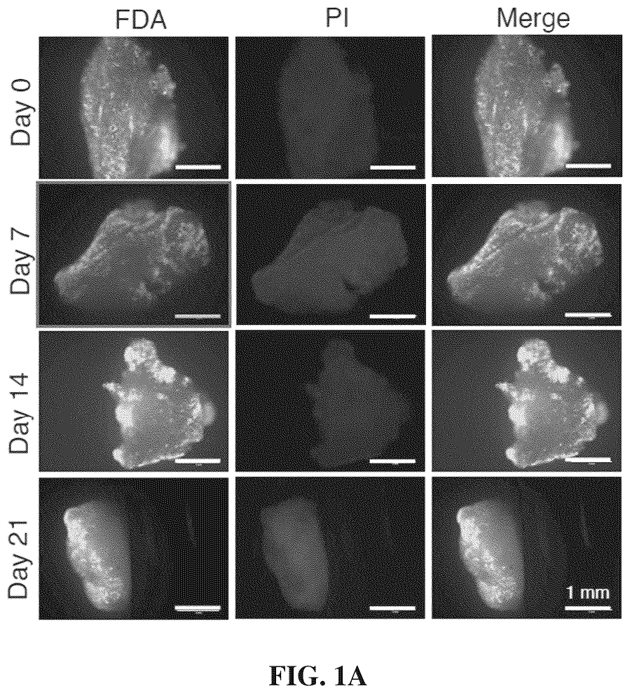

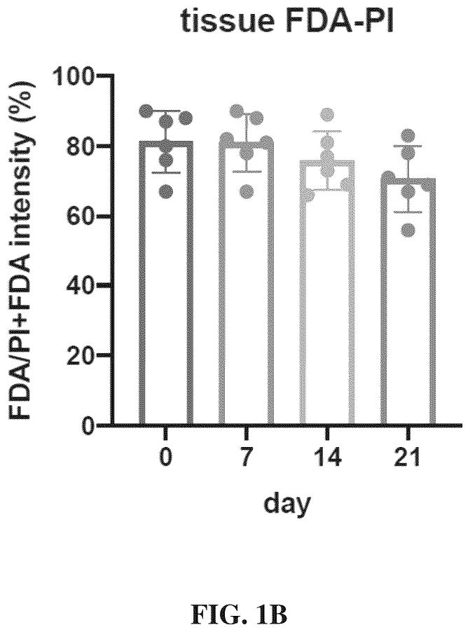

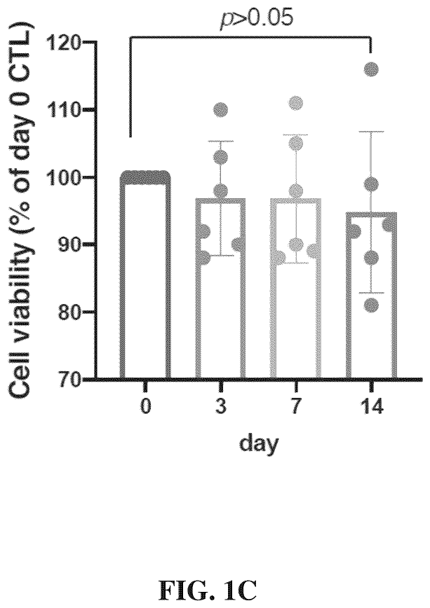

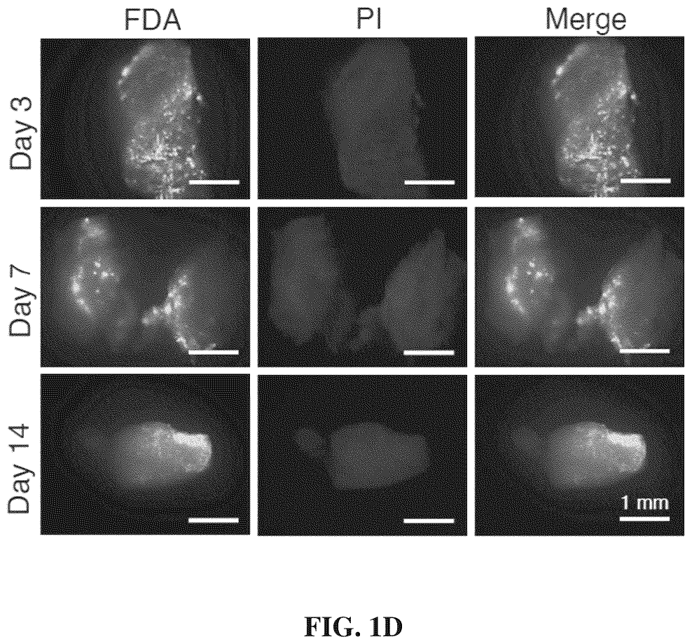

To confirm the viability of the parathyroid gland tissues in long-termed culture in vitro, the fresh human PTH glands collected from CMU hospital were dissected and filtrated by an 8-mesh filter, followed by live-and-dead staining. The FDA fluorescent counts over FDA+PI fluorescent counts were represented as viability. Data reveals the PTH tissue pieces with apparent FDA signals and slight PI fluorescence from day 0 to 21 (FIG. 1A). The FDA/PI+FDA signals intensity reached 70.67% at day 21, showing no significant difference in comparison with tissue pieces stained at day 0 (FIG. 1B). Meanwhile, the dissociated PTH cells were also subjected to MTT assay to examine the viability at long-termed culture in vitro; data shows the cells reached 94.83% viability at day 14 with no significant difference compared with cells at day 0 (FIG. 1C). Furthermore, to examine whether the calcium ion influences PTH tissue viability, the dissected tissue pieces were immersed in a completed medium containing one mM CaCl₂) for two weeks, data shows that PTH tissues maintained high viability (FIG. 1D).

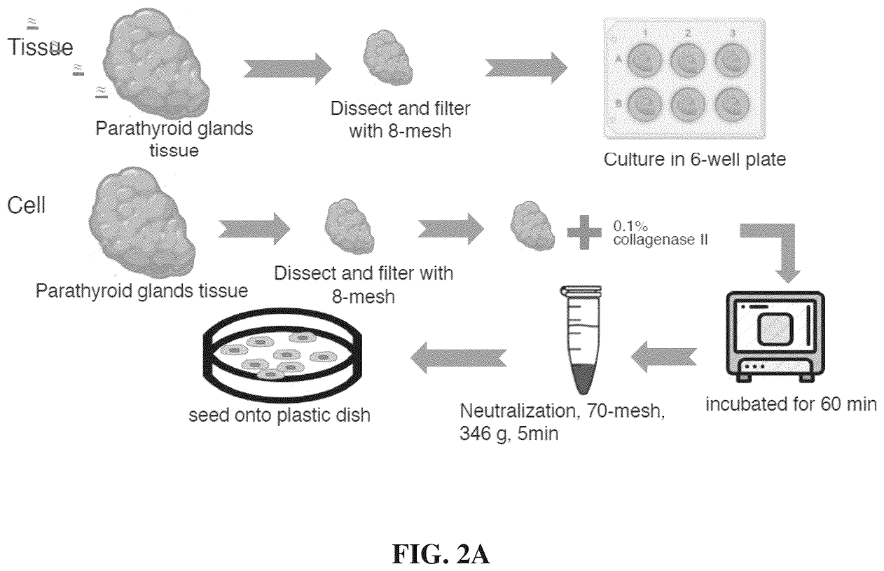

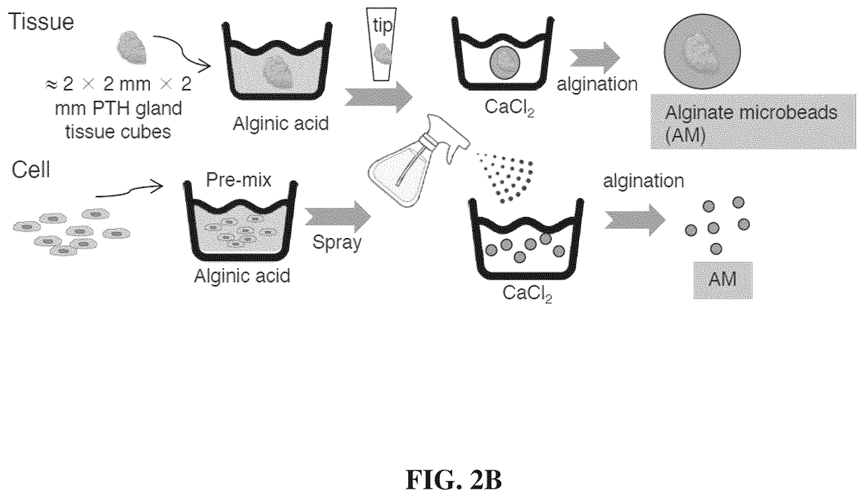

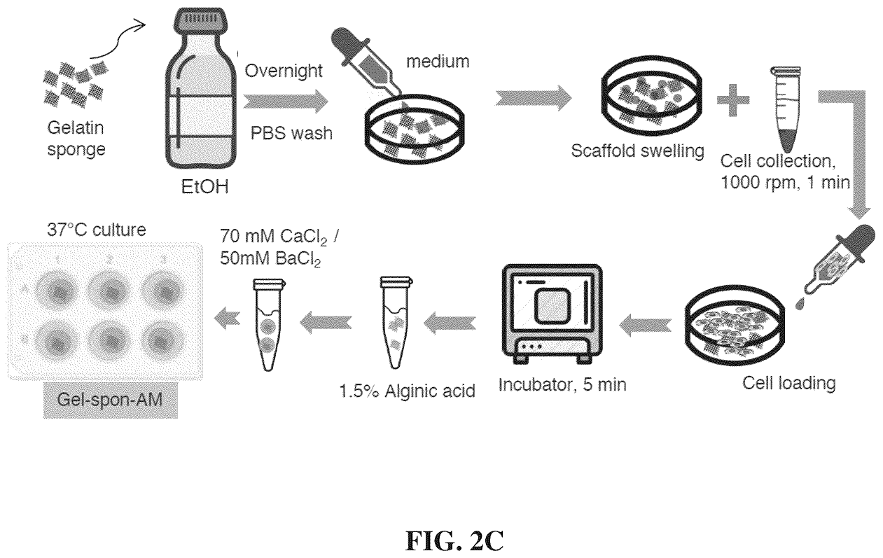

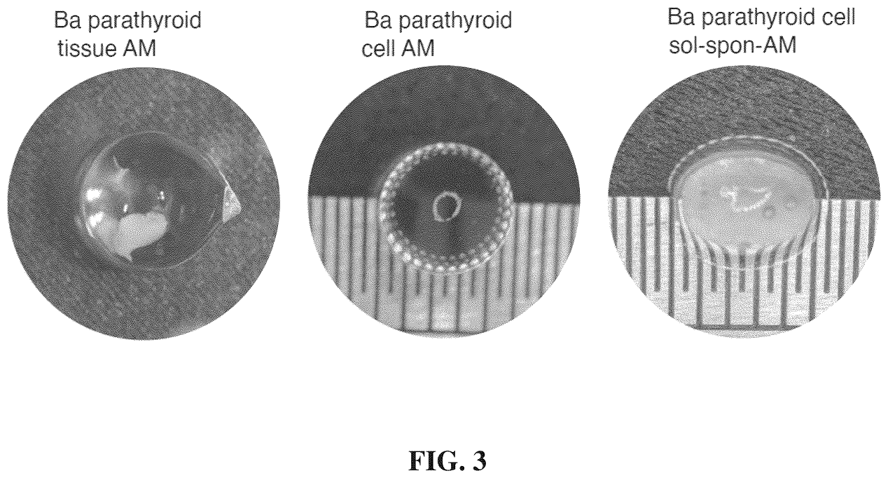

To compare the calcium-responsive PTH-releasing capability, PTH gland tissues were dissected and meshed into tiny pieces or dissociated by type II collagenase (FIG. 2A). Moreover, the PTH gland tissue pieces or PTH gland cells were encapsulated by alginate beads through calcium or barium ions, which assisted in gelation (FIG. 2B). Data shows that the PTH gland cells loaded alginate beads group hold superior calcium ions responsive PTH releasing capability than the PTH gland tissue pieces loaded alginate beads group at least two-fold. Furthermore, to increase the cell proliferation capability and maintain the PTH-releasing function, we developed a methodology, pre-loaded PTH grand cells in a gelatin sponge. Further, we encapsulated as an alginate bead, so-called “gelatin sponge-alginate composite microbeads (gel-spon-AM),” used for comparison with conventional method prepared alginate microbeads (AM) (FIG. 2C). The gross pictures of PTH gland tissue pieces loaded AM, PTH gland cells loaded AM, and PTH gland cells loaded as gel-spon-AM show approximately sphere shape and size (FIG. 3), which were used for subsequent experiments.

Result 2: Alginate Beads Crosslinked by Barium Ions Reveal Superior Mechanical Properties than Calcium Ions

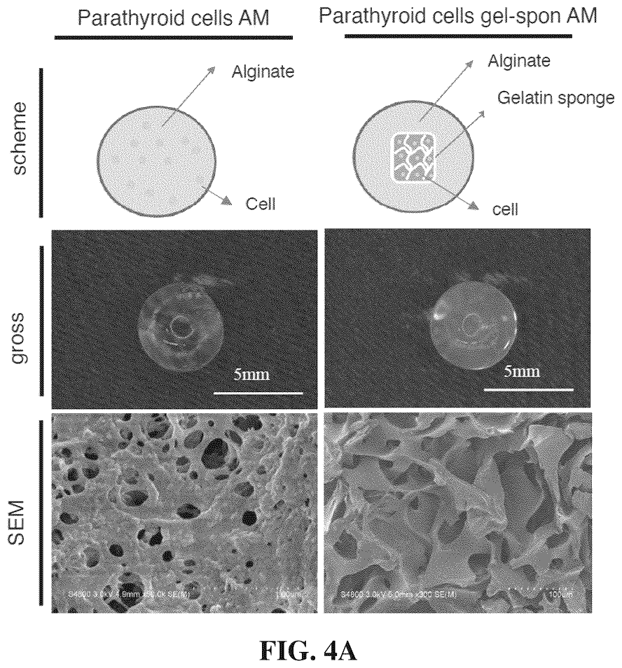

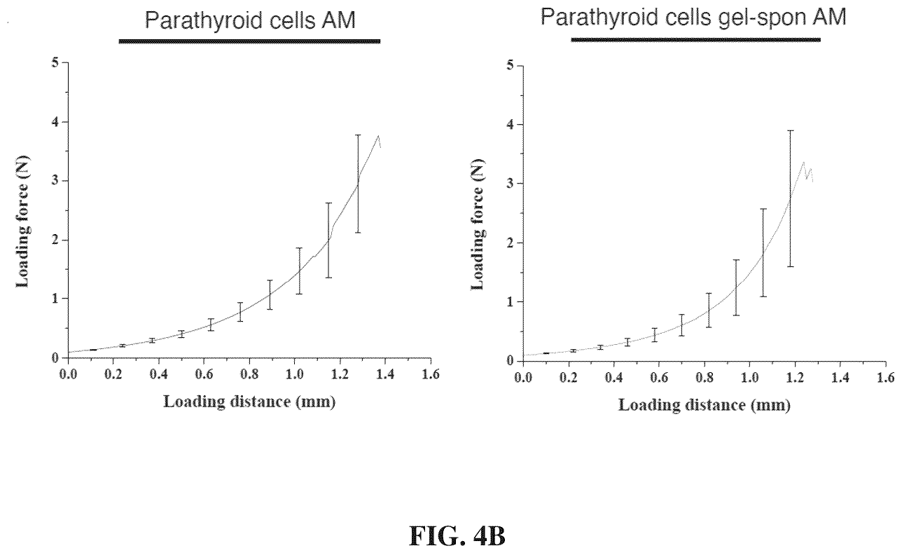

To examine the mechanical properties, the PTH gland cells loaded AM and PTH gland cells loaded gel-spon-AM were used for comparison. An axial force was loaded onto the microbeads till they thoroughly broke the alginate microbeads. The PTH gland cells loaded AM prepared by either calcium or barium ion showed equivalent biomechanical properties. However, the PTH gland cells loaded gel-spon-AM crosslinked by barium ion showed superior biomechanical properties than those crosslinked by calcium ion. Therefore, barium ions were used to prepare cell-loaded microbeads in the subsequent experiments. Data shows the gross pictures and SEM examinations of the barium ions crosslinked PTH cells loaded AM and gel-spon-AM (FIG. 4A), revealing the uniform sphere shape and connected channel in the microbeads. In the breaking test of microbeads, data shows the PTH cells AM prepared by barium crosslinking possess slightly higher compressing force, reaching 3.75 N and 1.38 mm. Meanwhile, the PTH cells gel-spon AM prepared by barium crosslinking shows a maximum compressing force of 3.37 N and 1.23 mm (FIG. 4B).

Result 3: Parathyroid Cells-Loaded Gelatin Sponge-Alginate Composite Microbeads (Gel-Spon-AM) Reveal Higher Calcium-Responsive PTH Secretory Capability than Conventional Alginate Microbeads (AM)

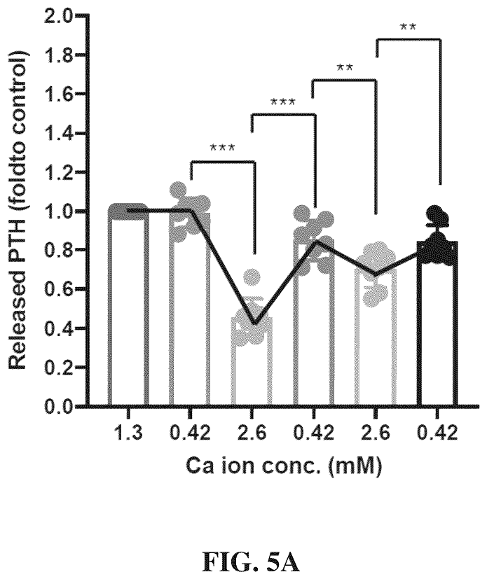

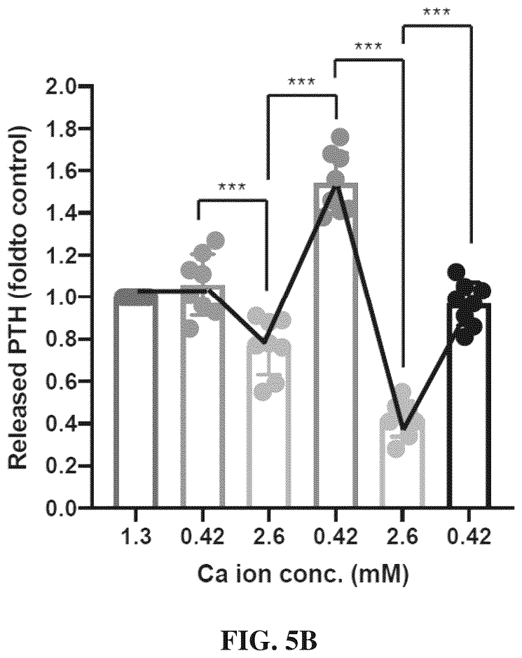

To examine the capability of parathyroid cells loaded AM or gel-spon-AM to sense the concentration changing of surrounding calcium ion to secrete the PTH, both types of microbeads were sequentially immersed in RPMI 1640 medium containing 1.3 mM, 0.42 mM, 2.6 mM, 0.42 mM, 2.6 mM, and 0.42 mM CaCl₂) for 30 minutes, respectively. The medium was collected for PTH ELISA measurement. Data reveals that the PTH released from the parathyroid cells loaded AM slightly changed, followed by the calcium ion concentration adjusted, which showed a significant difference (FIG. 5A). Interestingly, the PTH released from the parathyroid cells loaded gel-spon-AM, obviously changed, followed with the calcium ion concentration adjusted. PTH secreted sharply increased over 101.14% in calcium concentration adjusted from 2.6 mM to 0.42 mM, meanwhile decreased over 72.59% in calcium concentration returned from 0.42 mM to 2.6 mM. Subsequently, PTH increased by over 128.99% in calcium concentration, adjusted from 2.6 mM to 0.42 mM (FIG. 5B). The calcium-responsive PTH secretory capability is ameliorated in parathyroid cells loaded gel-spon-AM.

Result 4: Composite Gel-Spon-AM Provides Superior Parathyroid Cell Viability, Proliferation, and Quality than Conventional AM

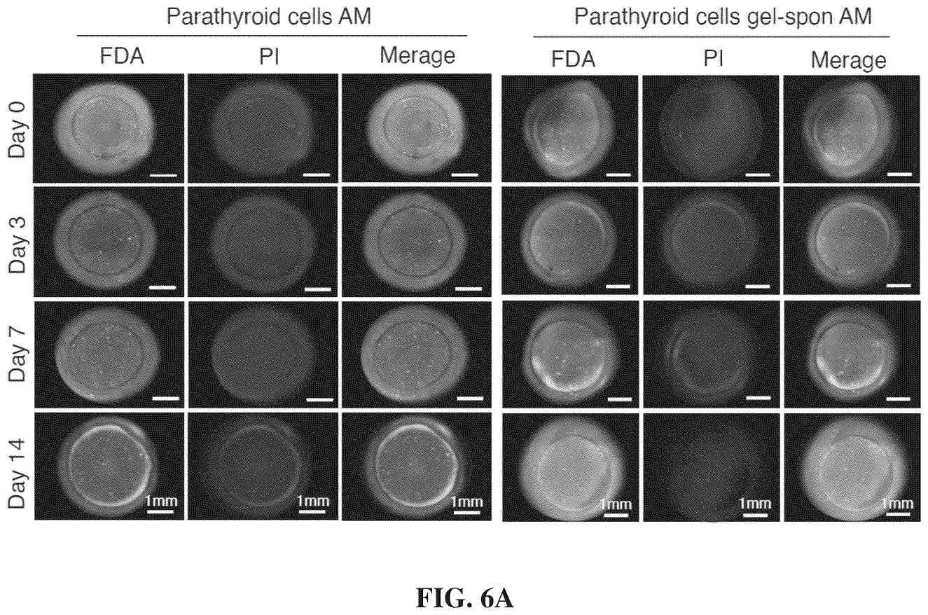

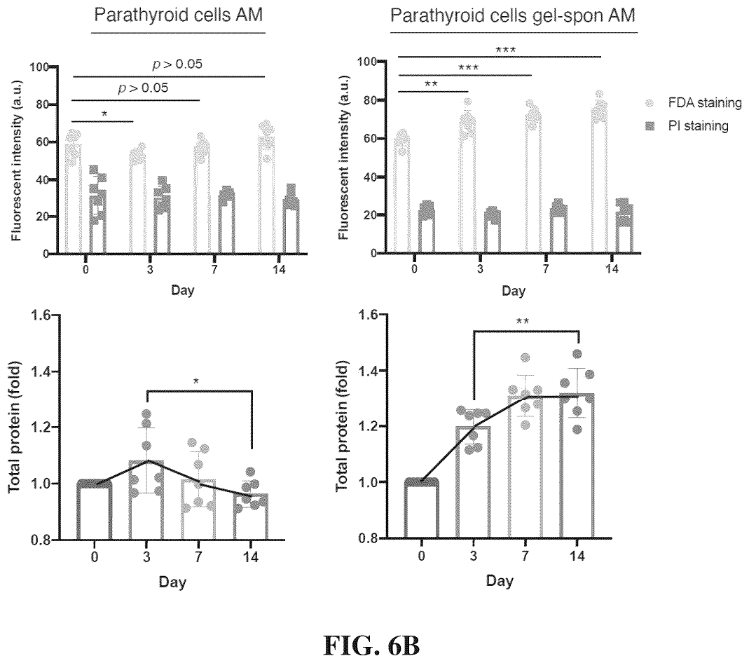

To assess the viability of parathyroid cells after being loaded in conventional AM and gel-spon-AM, the cells-packaged microbeads were stained with FDA and PI, respectively. The fluorescent intensity was statistically counted for comparison. The representative fluorescent images of microbeads stained with FDA and PI reveal that the FDA intensity in the gel-spon-AM group is stronger than the conventional AM group (FIG. 6A). The PI intensity represents the dead cell signals that show weak intensity. Furthermore, conventional AM's total FDA intensity from day 0 to 14 shows no statistical difference. Meanwhile, the PI intensity approximately reaches 30 arbitrary units (a.u.) on days 7 to 14 (FIG. 6B). Besides, the total cellular protein extracted from microbeads was examined, reveals the cellular protein reached the maximum at day three and gradually decreased to day 14 with a statistical difference. Conversely, the total FDA intensity in the gel-spon-AM group gradually increased with an extended culture period, reaching approximately 76 a.u. with low PI intensity near 20 a.u. at day 14 (FIG. 6B). Notably, the total cellular protein extracted from microbeads represents the proliferation scenario of the encapsulated cells, which increased with an extended culture period, reached the maximum at day 14, and shows significant differences in comparison with day 3.

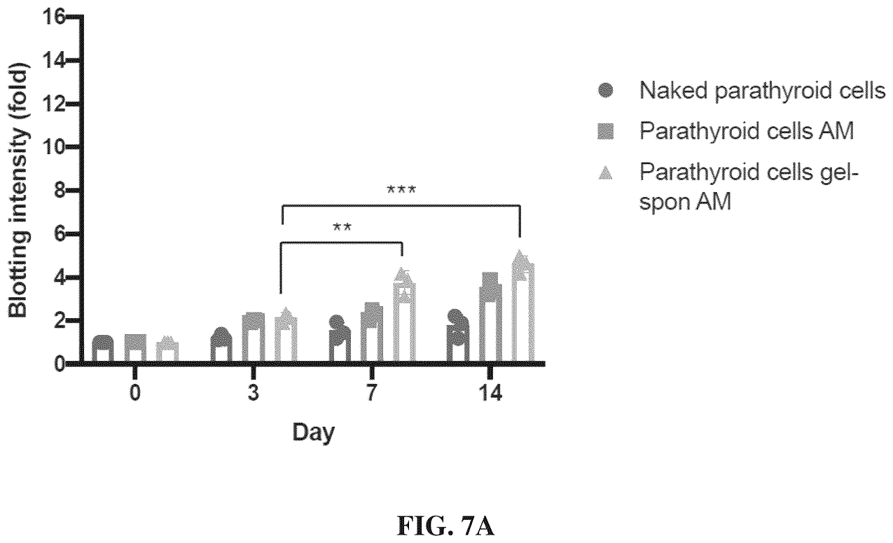

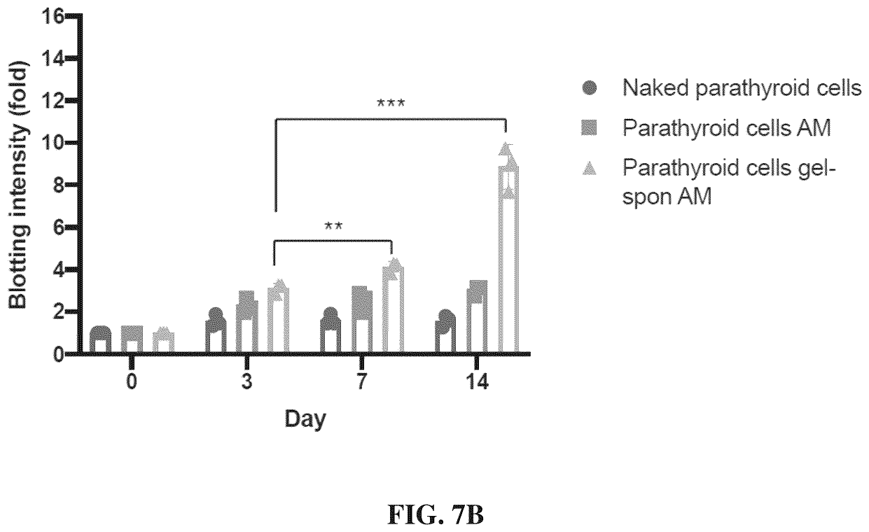

After the viability and proliferation of parathyroid cells loaded in microbeads were confirmed, the vital and critical cellular proteins, calcium-sensing receptor (CaSR) and Glial cells missing-2 (GCM2), were further examined to access the parathyroid cells' quality after encapsulated in microbeads and cultured in vitro. Naked cells without alginate encapsulation were compared with cells loaded in conventional AM or gel-spon-AM. All protein blots were normalized by internal control GAPDH and compared with proteins collected on day 0. Data shows CaSR protein expression gradually increased from day 0 to day 14 culture in all three groups. The CaSR protein increased in the gel-spon-AM group higher than in naked and conventional AM groups, and the increasing range at day seven and day 14 compared to day 3 revealed significant difference (FIG. 7A). Moreover, the GCM2 transcript factor increased in the gel-spon-AM group at day 7 and day 14, show significant difference in comparison with day 3 (FIG. 7B). Data demonstrates the parathyroid cells prepared by gel-spon-AM scenario provide cells with a superior viability, proliferation rate and physiological property.

### Result 5: Composite Gel-Spon-AM Exhibits Ameliorated Human Parathyroid Cell Protection Capability in the Xenotransplantation in a Mouse Model

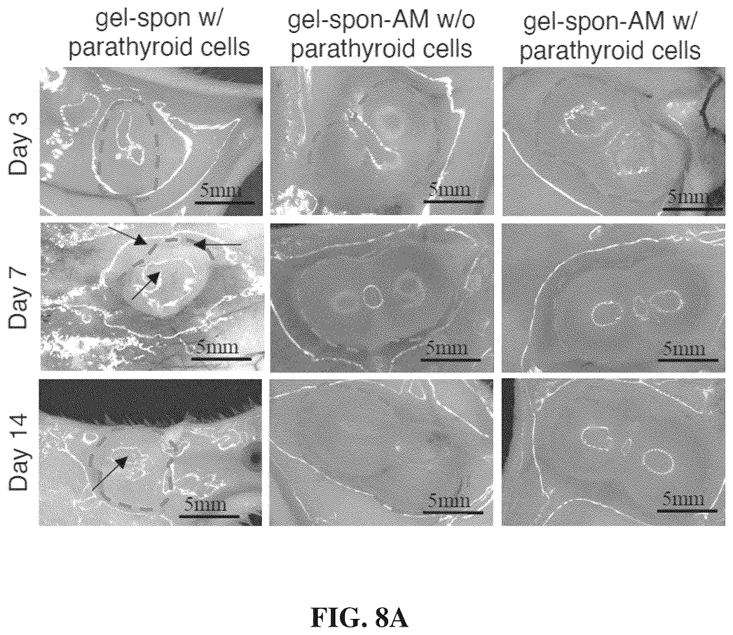

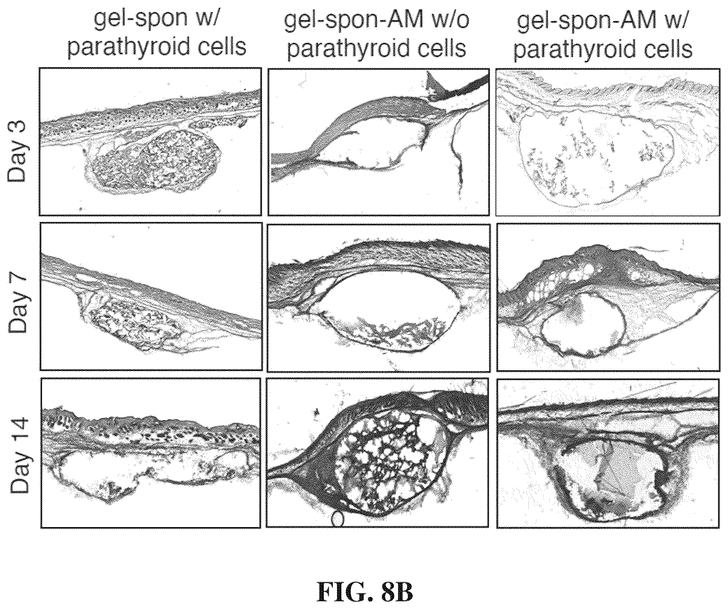

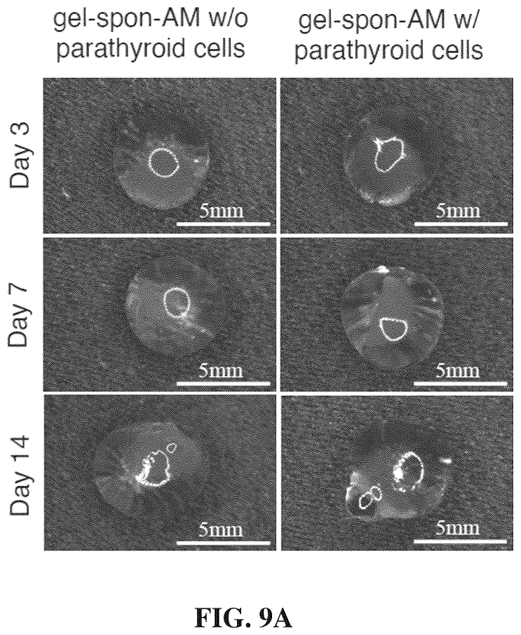

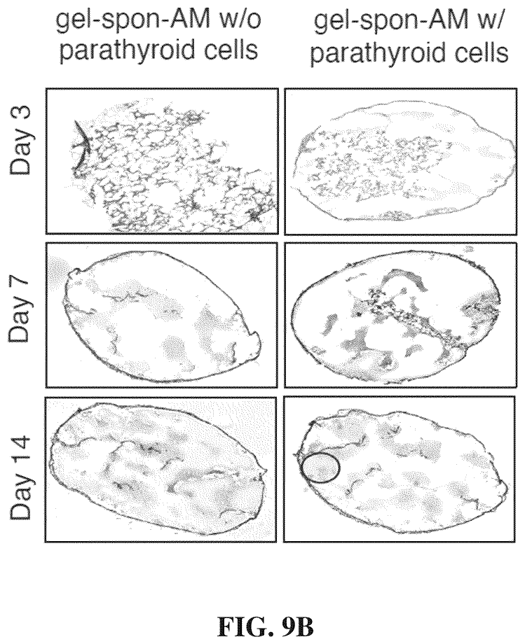

To assess the microbeads provide a compartment, and ameliorate the immune cells infiltration and foreign body reaction resulting from xenogeneic cell implantation, the human parathyroid cells loaded in the gelatin sponge with or without alginate encapsulation were subcutaneously implanted in the lateral backsides and intraperitoneally implanted in the peritoneal cavity under the parietal peritoneum and behind the diaphragm in mice, respectively. The microbeads were collected at day 3 to 14 post-implantation for histological evaluation. The gross pictures of the microbeads from subcutaneous implantation show apparent inflammation and soft-white inclusion body-like materials deposition in the parathyroid cells loaded in the gelatin sponge without alginate encapsulation, but no apparent inflammation and tissue swelling phenomenon in the gel-spon-AM with or without parathyroid cells loading (FIG. 8A). Meanwhile, apparent foreign body reaction and fibrous tissue infiltration in the parathyroid cells loaded in the gelatin sponge without alginate encapsulation (FIG. 8B). Conversely, the H&E staining shows clear microbeads' contour and sharp margin compared by alginate encapsulation without inflammatory cells infiltration in gel-spon-AM with or without parathyroid cells (FIG. 8B). Remarkably, the surrounding tissues in the gel-spon-AM with parathyroid cells xenotransplantation group, reveal no cell leakage resulted inflammation. Interestingly, the gelatin sponge without alginate encapsulation promptly degraded in the intraperitoneal implantation scenario, which led to no debris found in the gross observation. The representative gross pictures of gel-spon-AM with or without parathyroid cells show clear and transparent microbeads after retrieval from intraperitoneal implantation (FIG. 9A). Furthermore, the H&E staining shows clear microbeads' margin and no fibrous tissue infiltration in the both gel-spon-AM with or without parathyroid cells groups. Notably, there was no apparent foreign body reaction and immune cell attack in the gel-spon-AM with parathyroid cells xenotransplantation group (FIG. 9B).

### Result 6: Composite Gel-Spon-AM Diminishes Immune Cell Infiltration and Foreign Body Reaction in the Xenotransplantation Model

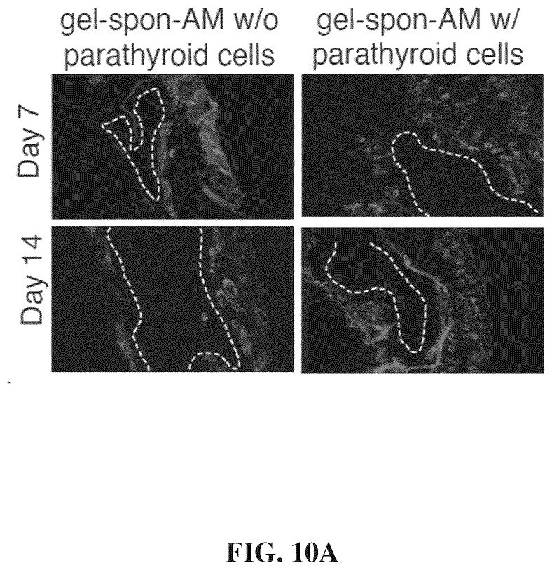

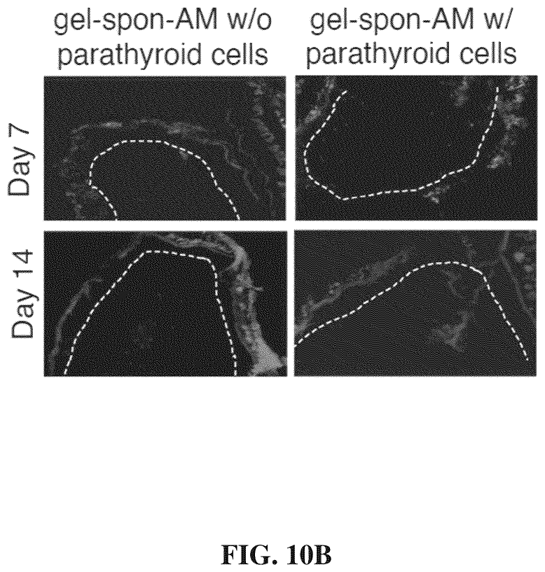

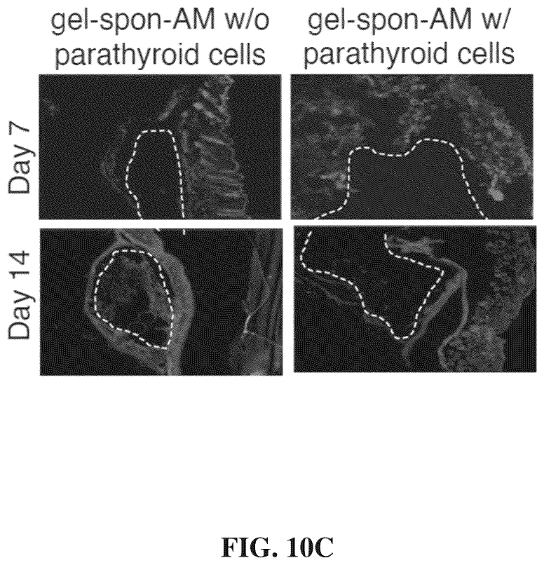

To examine the immune cells and fibrous tissue infiltration in the microbeads implantation site at a detailed molecular level, the CD⁴⁺ T-cell marker, CD68 circulating and tissue macrophage maker, and α-SMA fibroblast maker were examined in the tissue sections collected from microbeads post-implantation in vivo. Tissue sections retrieved from parathyroid cells loaded in gelatin sponge with or without alginate encapsulation groups were immunostained with primary antibodies against CD4, CD68, and α-SMA, compared with the sections retrieved from gel-spon-AM group without parathyroid cells loading. In the subcutaneous implantation, data shows apparent CD4 (FIG. 10A), CD68 (FIG. 10B), and α-SMA (FIG. 10C) positive signals in the group with parathyroid cells loaded gelatin sponge without alginate encapsulation. Significantly, both gel-spon-AM with or without parathyroid cells loading groups show almost no CD4 (FIG. 10A), CD68 (FIG. 10B), and α-SMA (FIG. 10C) signals and reveal a precise contour of the microbeads implantation site. Tissues surrounding the microbeads also reveal sparse CD⁴⁺ and CD⁶⁸⁺ immune cell aggregation, demonstrating the xenogeneic parathyroid cells safely encapsulated and compared in the microbeads. In the intraperitoneal implantation, both groups using gel-spon-AM with or without parathyroid cell loading were compared in parallel. Data shows the CD4, CD68, and α-SMA signals were almost none detectable in the microbeads, again demonstrating that the immune cells and fibroblasts were sophisticatedly constricted outside the microbeads.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A gel composition, comprising:
a porous scaffold filled with a biological tissue or a biological cell; and
a gel coating covering the porous scaffold.

2. The gel composition as claimed in claim 1, wherein the gel coating comprises one or more of agar, agarose, alginic acid, alguronic acid, α-glucan, amylopectin, amylose, arabinoxylan, β-glucan, callose, carrageenan polysaccharides, cellodextrin, cellulin, cellulose, chitin, chitosan, chrysolaminarin, curdlan, cyclodextrin, α-cyclodextrin, dextrin, glucan, ficoll, fructan, fucoidan, galactoglucomannan, galactomannan, galactosaminoogalactan, gellan gum, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, schizophyllan, sinistrin, sizofiran, welan gum, xanthan gum, xylan, xyloglucan, and zymosan.

3. The gel composition as claimed in claim 1, wherein the porous scaffold comprises one or more of a porous polymeric particle or matrix, a porous inorganic particle or matrix, and an etched metal particle or matrix.

4. The gel composition as claimed in claim 1, wherein the biological tissue comprises one or more of a pancreatic islet tissue, a pancreatic duct tissue, a liver tissue, a nervous tissue, a thyroid gland tissue, a parathyroid gland tissue, a kidney tissue, an adrenal gland tissue, a pituitary gland tissue, a spleen tissue, an adipose tissue, and a myeloid tissue.

5. The gel composition as claimed in claim 1, wherein the biological cell comprises one or more of a pancreatic islet cell, a pancreatic duct cell, a liver cell, a nervous cell, a thyroid gland cell, a parathyroid gland cell, a kidney cell, an adrenal gland cell, a pituitary gland cell, a spleen cell, an adipose cell, and a myeloid cell.

6. The gel composition as claimed in claim 2, wherein the gel coating comprises alginic acid.

7. The gel composition as claimed in claim 3, wherein the porous polymeric particle or matrix comprises one or more of chitosan, hyaluronic acid, gelatin, collagen, chitosan, poly(lactic-co-glycolic acid) (PLGA), polycyclohexylenedimethylene terephthalate (PCT), the porous inorganic particle or matrix comprises one or more of silicon dioxide and tricalcium phosphate, and the etched metal particle or matrix comprises titanium.

8. The gel composition as claimed in claim 3, wherein the porous polymeric particle or matrix comprises gelatin.

9. The gel composition as claimed in claim 4, wherein the biological tissue comprises a parathyroid gland tissue.

10. The gel composition as claimed in claim 4, wherein the biological cell comprises a parathyroid gland cell.

11. The gel composition as claimed in claim 1, wherein the gel coating comprises alginic acid, the porous scaffold comprises gelatin, the biological tissue comprises a parathyroid gland tissue, and the biological cell comprises a parathyroid gland cell.

12. A method for producing the gel composition as claimed in claim 1, comprising:
filling the porous scaffold with the biological tissue or the biological cell;
immersing the porous scaffold in a gel precursor solution; and
adding a crosslinker solution into the gel precursor solution immersed with the porous scaffold to perform cross-linking reaction so as to form the gel coating, the gel coating covering the porous scaffold.

13. The method as claimed in claim 12, wherein the crosslinker solution comprises one or more of a calcium ion, a barium ion, and a zinc ion.

14. The method as claimed in claim 12, wherein the crosslinker solution comprises a barium ion.

15. The method as claimed in claim 12, wherein before filling the porous scaffold with the biological tissue or the biological cell, the biological tissue or the biological cell is digested or dissociated with an enzyme.

16. The method as claimed in claim 15, wherein the enzyme is a type II collagenase.

17. A method for xenotransplantation or allotransplantation, comprising:
implanting the gel composition as claimed in claim 1 into a subject in need thereof.

18. A method for xenotransplantation or allotransplantation, comprising:
implanting the gel composition as claimed in claim 11 into a subject in need thereof.
